(19) United States
(12) Patent Application Publication
(10) Pub. No.: US 2017/0365634 A1
(43) Pub. Date: Dec. 21, 2017

(54) IMAGE SENSOR AND IMAGING DEVICE

(71) Applicant: OLYMPUS CORPORATION, Tokyo (JP)

(72) Inventors: Jun AOKI, Tokyo (JP); Satoru ADACHI, Tsuchiura-shi (JP)

(73) Assignee: OLYMPUS CORPORATION, Tokyo (JP)

(21) Appl. No.: 15/690,339

(22) Filed: Aug. 30, 2017

Related U.S. Application Data
(63) Continuation of application No. PCT/JP2016/062037, filed on Apr. 14, 2016.

(30) Foreign Application Priority Data
Sep. 30, 2015 (JP) 2015-193871

(51) Int. Cl. H01L 27/146; A61B 1/05; H04N 9/077; H04N 9/04; H04N 5/225; A61B 1/00; A61B 1/06

(52) U.S. Cl. CPC H01L 27/14621; H04N 5/2256; A61B 1/05; A61B 1/00186; H04N 9/077; H04N 9/045; H01L 27/14627; A61B 1/0684; H04N 2005/2255

(57) ABSTRACT

An image sensor includes: light receiving units disposed two-dimensionally on a substrate; color filters disposed on the light receiving units and including at least one of: a blue color filter for passing both of blue light and blue-violet light; a cyan color filter for passing both of green light and the blue-violet light; and a magenta color filter for passing both of red light and the blue-violet light; a first film arranged on a light receiving unit on which the cyan color filter is disposed, among the light receiving units, the first film having a peak of reflectivity near 450 nm; and a second film arranged on a light receiving unit on which the magenta color filter is disposed, among the light receiving units, the second film having a peak of reflectivity between 450 nm and 500 nm.

### CROSS REFERENCES TO RELATED APPLICATIONS

This application is a continuation of PCT international application Ser. No. PCT/JP2016/062037, filed on Apr. 14, 2016 which designates the United States, incorporated herein by reference, and which claims the benefit of priority from Japanese Patent Application No. 2015-193871, filed on Sep. 30, 2015, incorporated herein by reference.

### BACKGROUND

### 1. Technical Field

The disclosure relates to an image sensor and an imaging device.

### 2. Related Art

Conventionally, normal light imaging for emitting normal light (white light) to an observation region, and narrow band imaging (NBI) for emitting narrow band light in a predetermined wavelength band to an observation region are known as observation methods in endoscope systems. The narrow band light used for NBI is NBI illumination light including green light (with a wavelength of 540 nm, for example) and blue-violet light (with a wavelength of 410 nm, for example) whose wavelength band is narrow enough to be easily absorbed into hemoglobin in blood. The NBI provides enhanced imaging of capillaries and mucosal patterns on mucosal surface layers of a living body (surface layers of a living body).

A primary color image sensor including a primary color filter, and a complementary color image sensor using a complementary color filter are known as image sensors used for an endoscope system. The primary color filter is a color filter for passing light in a wavelength band of each of red (R), green (G), and blue (B). The complementary color filter is a color filter for passing light in a wavelength band of each of cyan (Cy), magenta (Mg), yellow (Ye), and green (G).

If a primary color image sensor is used in NBI, R and G pixels that respectively include R and G color filters do not have sensitivity for light in a wavelength band of blue-violet of NBI illumination light. Thus, only a B pixel including a B color filter can be used in NBI and resolution is not good. Thus, a technology of improving resolution by using a complementary color image sensor in NBI has been disclosed (see JP 2015-66132 A, for example).

### SUMMARY

In some embodiments, an image sensor includes: a plurality of light receiving units disposed two- dimensionally on a substrate and each configured to generate a charge in accordance with an amount of received light; color filters disposed on the plurality of light receiving units and including at least one of: a blue color filter for passing both of light in a wavelength band of blue and light in a wavelength band of blue-violet; a cyan color filter for passing both of light in a wavelength band of green and light in the wavelength band of blue-violet; and a magenta color filter for passing both of light in a wavelength band of red and light in the wavelength band of blue-violet; a first film arranged on a light receiving unit on which the cyan color filter is disposed, among the plurality of light receiving units, the first film having a peak of reflectivity near 450 nm; and a second film arranged on a light receiving unit on which the magenta color filter is disposed, among the plurality of light receiving units, the second film having a peak of reflectivity between 450 nm and 500 nm.

In some embodiments, an imaging device includes the image sensor.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic view illustrating a configuration of a whole endoscope system including an imaging device according to an embodiment of the present invention;

FIG. 2 is a block diagram illustrating a function of a main part of the endoscope system according to the embodiment of the present invention;

FIG. 3 is a schematic view illustrating a configuration of a color filter according to the embodiment of the present invention;

FIG. 4 is a sectional view of a B pixel;

FIG. 5 is a sectional view of a Cy pixel;

FIG. 6 is a schematic view illustrating sensitivity of an element including the Cy color filter;

FIG. 7 is a sectional view of an Mg pixel; and

FIG. 8 is a schematic view illustrating sensitivity of an element including an Mg color filter.

### DETAILED DESCRIPTION

As exemplary embodiments of the present invention, reference will be made to an endoscope system including an endoscope a distal end of which is configured to be inserted into a subject. The present invention is not limited to the embodiments. The same reference signs are used to designate the same elements throughout the drawings. The drawings are schematic and a relationship between a thickness and a width of each member, a proportion of each member, and the like are different from the reality. The drawings may include parts with sizes or proportions being different from each other.

### Configuration of Endoscope System

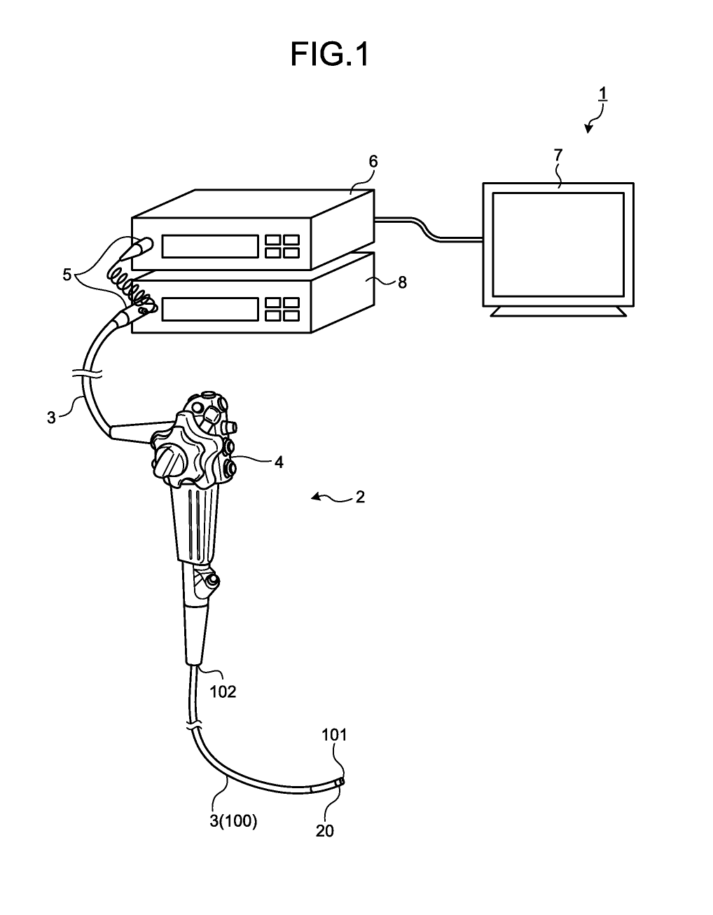

FIG. 1 is a schematic view illustrating a configuration of a whole endoscope system including an imaging device according to an embodiment of the present invention. An endoscope system 1 illustrated in FIG. 1 includes an endoscope 2, a transmission cable 3, an operating unit 4, a connector unit 5, a processor 6 (processing device), a display device 7, and a light source device 8.

The endoscope 2 includes an insertion unit 100, as a part of the transmission cable 3, to be inserted into a body cavity of a subject to capture images, and outputs an imaging signal (image data) to the processor 6. The endoscope 2 includes an imaging unit 20 (imaging device) for capturing in-vivo images on one end of the transmission cable 3 and at a distal end 101 of the insertion unit 100 configured to be inserted into the body cavity of the subject, and includes an operating unit 4 at a proximal end 102 of the insertion unit 100 to receive various kinds of operation with respect to the endoscope 2. The imaging signal of images captured by the imaging unit 20 is output to the connector unit 5, for example, through the transmission cable 3 having a length of a several meters.

The transmission cable 3 connects the endoscope 2 and the connector unit 5, and connects the endoscope 2 and the light source device 8. The transmission cable 3 transmits the imaging signal generated by the imaging unit 20 to the connector unit 5. The transmission cable 3 includes a cable, an optical fiber, or the like.

The connector unit 5 is connected to the endoscope 2, the processor 6, and the light source device 8, performs predetermined signal processing on an imaging signal output by the connected endoscope 2, converts an analog imaging signal into a digital imaging signal (perform A/D conversion), and outputs the digital imaging signal to the processor 6.

The processor 6 performs predetermined image processing on the imaging signal input from the connector unit 5, and outputs the imaging signal to the display device 7. The processor 6 further performs overall control of the endoscope system 1. For example, the processor 6 switches illumination light emitted by the light source device 8 and switches between imaging modes of the endoscope 2.

The display device 7 displays an image corresponding to the imaging signal after the image processing by the processor 6. Also, the display device 7 displays various kinds of information on the endoscope system 1. The display device 7 includes a liquid-crystal or organic electro luminescence (EL) display panel, or the like.

The light source device 8 emits illumination light toward an object from the distal end 101 of the insertion unit 100 of the endoscope 2 via the connector unit 5 and the transmission cable 3. The light source device 8 includes a white light emitting diode (LED) for emitting white light and an LED for emitting special light in a narrow band (NBI illumination light) having a wavelength band narrower than a wavelength band of the white light. The light source device 8 emits the white light or NBI illumination light to an object via the endoscope 2 under control of the processor 6. The light source device 8 employs simultaneous lighting in the embodiments.

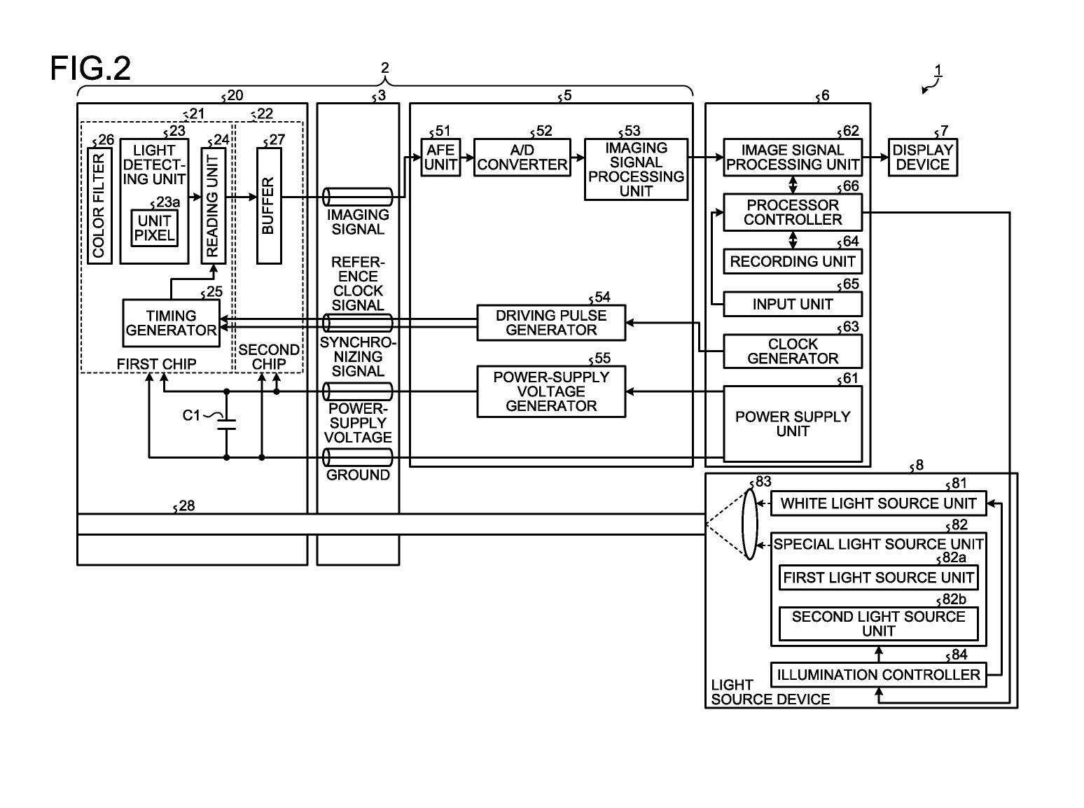

FIG. 2 is a block diagram illustrating a function of a main part of the endoscope system according to the embodiment of the present invention. A detail of a configuration of each unit of the endoscope system 1, and a channel of an electric signal in the endoscope system 1 will be described with reference to FIG. 2.

### Configuration of Endoscope

First, a configuration of the endoscope 2 will be described. The endoscope 2 illustrated in FIG. 2 includes an imaging unit 20, a transmission cable 3, and a connector unit 5.

The imaging unit 20 includes a first chip 21 (image sensor) and a second chip 22. The imaging unit 20 receives a power-supply voltage VDD, which is generated by a power supply unit 61 of the processor 6, along with a ground GND through the transmission cable 3. A capacitor Cl for power-supply stabilization is provided between the power-supply voltage VDD and the ground GND, which are supplied to the imaging unit 20.

The first chip 21 includes a light detecting unit 23 in which a plurality of unit pixels 23a that is arranged in a two-dimensional matrix, that receives light from the outside, and that generates and outputs an image signal corresponding to an amount of received light is arranged, a reading unit 24 that reads an imaging signal photoelectrically converted in each of the plurality of unit pixels 23a of the light detecting unit 23, a timing generator 25 that generates a timing signal on the basis of a reference clock signal and a synchronizing signal input from the connector unit 5 and outputs these signals to the reading unit 24, and a color filter 26 arranged on a light receiving surface of each of the plurality of unit pixels 23a.

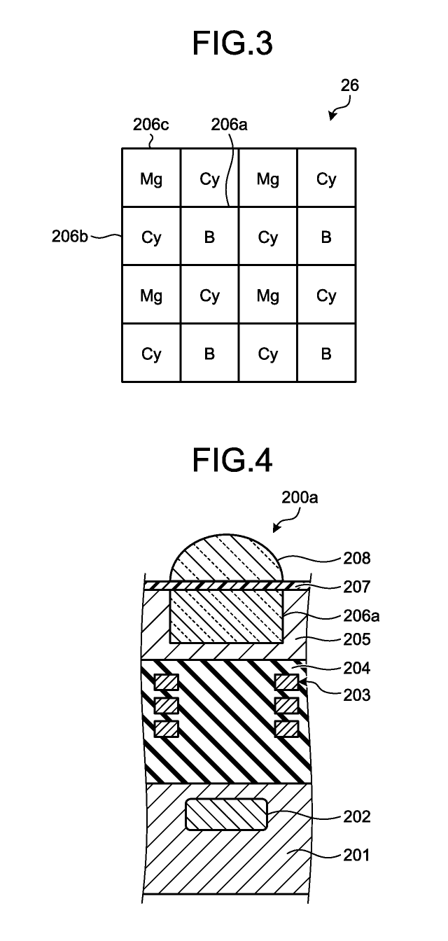

FIG. 3 is a schematic view illustrating a configuration of a color filter according to the embodiment of the present invention. As illustrated in FIG. 3, in the color filter 26, with respect to a color filter in a Bayer array including RGB color filters, a B color filter is arranged at a position corresponding to a B color filter in the Bayer array, a Cy color filter is arranged at a position corresponding to a G color filter in the Bayer array, and an Mg color filter is arranged at a position corresponding to an R color filter in the Bayer array. More specifically, in the color filter 26, a Cy color filter 206b and a B color filter 206a are alternately arranged in an even number line in horizontal lines of a plurality of light receiving units, and an Mg color filter 206c and a Cy color filter 206b are alternately arranged in an odd number line in the horizontal lines of the plurality of light receiving units. In the following, a unit pixel 23a on which the B color filter 206a is disposed is referred to as a B pixel 200a, a unit pixel 23a on which the Cy color filter 206b is disposed is referred to as a Cy pixel 200b, and a unit pixel 23a on which the Mg color filter 206c is disposed is referred to as an Mg pixel 200c. That is, the endoscope system 1 has a configuration in which a G pixel in the Bayer array is replaced with the Cy pixel 200b and an R pixel in the Bayer array is replaced with the Mg pixel 200c. The more detailed description of a pixel in each color will be made later.

Referring back to FIG. 2, the second chip 22 includes a buffer 27 that amplifies an imaging signal output from each of the plurality of unit pixels 23a in the first chip 21 and outputs the imaging signal to the transmission cable 3. The combination of circuits arranged in the first chip 21 and the second chip 22 can be arbitrarily changed. For example, the timing generator 25 arranged in the first chip 21 may be arranged in the second chip 22.

A light guide 28 emits illumination light, which is emitted from the light source device 8, toward an object. The light guide 28 is realized with a fiberglass, an illumination lens, or the like.

The connector unit 5 includes an analog front-end unit 51 (hereinafter, referred to as “AFE unit 51”), an A/D converter 52, an imaging signal processing unit 53, a driving pulse generator 54, and a power-supply voltage generator 55.

The AFE unit 51 receives the imaging signal transmitted from the imaging unit 20, performs impedance matching by using a passive element such as a resistor, and then, extracts an AC component by using a capacitor, and determines an operating point by a voltage dividing resistor. Subsequently, the AFE unit 51 corrects the imaging signal (analog signal) and outputs the analog imaging signal to the A/D converter 52.

The A/D converter 52 converts the analog imaging signal input from the AFE unit 51 into a digital imaging signal, and outputs the digital imaging signal to the imaging signal processing unit 53.

The imaging signal processing unit 53 includes, for example, a field programmable gate array (FPGA) to perform processing, such as noise elimination and format conversion, on the digital imaging signal input from the A/D converter 52, and outputs the imaging signal to the processor 6.

The driving pulse generator 54 generates a synchronizing signal indicating a start position of each frame on the basis of a reference clock signal (such as clock signal of 27 MHz), which is supplied from the processor 6 and which is a reference of an operation of each unit of the endoscope 2, and outputs the synchronizing signal along with the reference clock signal to the timing generator 25 of the imaging unit 20 through the transmission cable 3. Here, the synchronizing signal generated by the driving pulse generator 54 includes a horizontal synchronizing signal and a vertical synchronizing signal.

The power-supply voltage generator 55 generates a power-supply voltage for driving the first chip 21 and the second chip 22 from the power supplied from the processor 6, and outputs the power-supply voltage to the first chip 21 and the second chip 22. The power-supply voltage generator 55 uses a regulator or the like to generate the power- supply voltage for driving the first chip 21 and the second chip 22.

### Configuration of Processor

Next, a configuration of the processor 6 will be described.

The processor 6 is a control device to perform overall control of the endoscope system 1. The processor 6 includes a power supply unit 61, an image signal processing unit 62, a clock generator 63, a recording unit 64, an input unit 65, and a processor controller 66.

The power supply unit 61 generates a power-supply voltage VDD, and supplies the generated power-supply voltage VDD along with a ground GND to the imaging unit 20 via the connector unit 5 and the transmission cable 3.

The image signal processing unit 62 converts a digital imaging signal, on which signal processing is performed in the imaging signal processing unit 53, into an image signal by performing image processing such as synchronization processing, white balance (WB) adjustment processing, gain adjustment processing, gamma correction processing, digital analog (D/A) conversion processing, and format conversion processing with respect thereto, and outputs this image signal to the display device 7.

The clock generator 63 generates a reference clock signal to be a reference of an operation of each configuration unit of the endoscope system 1, and outputs this reference clock signal to the driving pulse generator 54.

The recording unit 64 records various kinds of information related to the endoscope system 1, currently- processed data, and the like. The recording unit 64 includes a recording medium such as a flash memory or a random access memory (RAM).

The input unit 65 receives an input of various kinds of operation related to the endoscope system 1. For example, the input unit 65 receives an input of a command signal for switching types of illumination light emitted by the light source device 8. The input unit 65 includes, for example, a four directional switch or a push button.

The processor controller 66 performs overall control of each unit of the endoscope system 1. The processor controller 66 includes a central processing unit (CPU). The processor controller 66 switches illumination light emitted by the light source device 8 according to a command signal input from the input unit 65.

### Configuration of Light Source Device

Next, a configuration of the light source device 8 will be described. The light source device 8 includes a white light source unit 81, a special light source unit 82, a condenser lens 83, and an illumination controller 84.

The white light source unit 81 emits white light toward the light guide 28 via the condenser lens 83 under control of the illumination controller 84. The white light source unit 81 includes a white light emitting diode (LED). The white light source unit 81 includes a white LED in the present embodiment. However, white light may be emitted, for example, by a xenon lamp or a combination of a red LED, a green LED, and a blue LED.

The special light source unit 82 simultaneously emits two rays of narrow band light (NBI illumination light) in different wavelength bands toward the light guide 28 via the condenser lens 83 under control of the illumination controller 84. The special light source unit 82 includes a first light source unit 82a and a second light source unit 82b.

The first light source unit 82a includes a blue-violet LED. The first light source unit 82a emits narrow band light in a band narrower than a wavelength band of blue under control of the illumination controller 84. More specifically, the first light source unit 82a emits light in a wavelength band of blue-violet in the vicinity of 410 nm (such as 390 nm to 440 nm) under control of the illumination controller 84.

The second light source unit 82b includes a green LED. The second light source unit 82b emits narrow band light in a band narrower than a wavelength band of green under control of the illumination controller 84. More specifically, the second light source unit 82b emits light in a wavelength band of green in the vicinity of 540 nm (such as 530 nm to 550 nm) under control of the illumination controller 84.

The condenser lens 83 collects the white light emitted by the white light source unit 81 or the NBI illumination light emitted by the special light source unit 82, and performs emission thereof to the light guide 28. The condenser lens 83 includes one or a plurality of lenses.

The illumination controller 84 controls the white light source unit 81 and the special light source unit 82 under control of the processor controller 66. More specifically, the illumination controller 84 makes the white light source unit 81 emit white light or makes the special light source unit 82 emit NBI illumination light under control of the processor controller 66. Also, the illumination controller 84 controls emission timing at which the white light source unit 81 emits white light or emission timing at which the special light source unit 82 emits NBI illumination light.

### Configuration of Pixel in Each Color

Next, a pixel in each color will be described in detail. First, a B pixel will be described. FIG. 4 is a sectional view of a B pixel. As illustrated in FIG. 4, a B pixel 200a includes an Si substrate 201, a photodiode 202 that is formed on the Si substrate 201 as a light receiving unit, a wiring layer 203 that electrically connects pixels, an insulator layer 204 that electrically insulates each wiring layer 203, a buffer layer 205 to planarize a surface, a B color filter 206a that is arranged so as to cover the photodiode 202, a protective layer 207 that protects a surface, and a microlens 208 formed on an outermost surface.

The Si substrate 201 is a substrate made of silicon (Si). However, a substrate is not necessarily made of Si.

The photodiode 202 is a photoelectric conversion element and generates a charge corresponding to an amount of received light. The photodiodes 202 are arranged two- dimensionally on a plane vertical to a layering direction as illustrated in FIG. 3.

The B color filter 206a is a color filter for passing light in a wavelength band of blue in the vicinity of 450 nm. Thus, the B pixel 200a detects light in the wavelength band of blue under a white light source and detects light in a wavelength band of blue-violet under an NBI illumination light source.

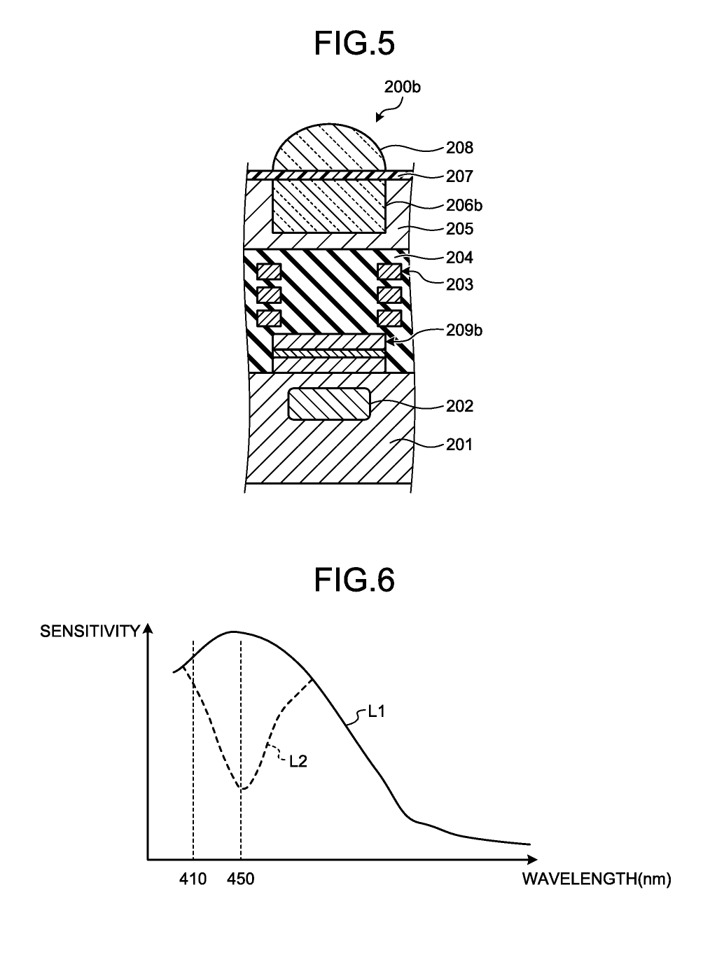

Next, a Cy pixel will be described. FIG. 5 is a sectional view of a Cy pixel. As illustrated in FIG. 5, a Cy pixel 200b includes an Si substrate 201, a photodiode 202 that is formed on the Si substrate 201, a wiring layer 203 that electrically connects pixels, an insulator layer 204 that electrically insulates each wiring layer 203, a buffer layer 205 to planarize a surface, a Cy color filter 206b that is arranged so as to cover the photodiode 202, a protective layer 207 that protects a surface, a microlens 208 formed on an outermost surface, and a Cy multi-layer film 209b as a first multi-layer film disposed on the Si substrate 201.

The Cy color filter 206b is a color filter for passing both of light in a wavelength band of green and light in a wavelength band of blue-violet.

The Cy multi-layer film 209b is a multi-layer film with a refractive index and a layer thickness of each layer being adjusted in such a manner that a peak of reflectivity is in the vicinity of 450 nm.

FIG. 6 is a schematic view illustrating sensitivity of an element including the Cy color filter. A line L1 in FIG. 6 indicates sensitivity of a conventional Cy pixel that includes a Cy color filter 206b and that does not include a Cy multi-layer film 209b. Then, a line L2 (broken line) in FIG. 6 indicates sensitivity of a Cy pixel 200b that includes a Cy color filter 206b and a Cy multi-layer film 209b. That is, under a white light source, light in a wavelength band of green is detected and sensitivity for light in a wavelength band of blue is weakened in the Cy pixel 200b. On the other hand, the Cy pixel 200b detects light in a wavelength band of blue-violet under an NBI illumination light source.

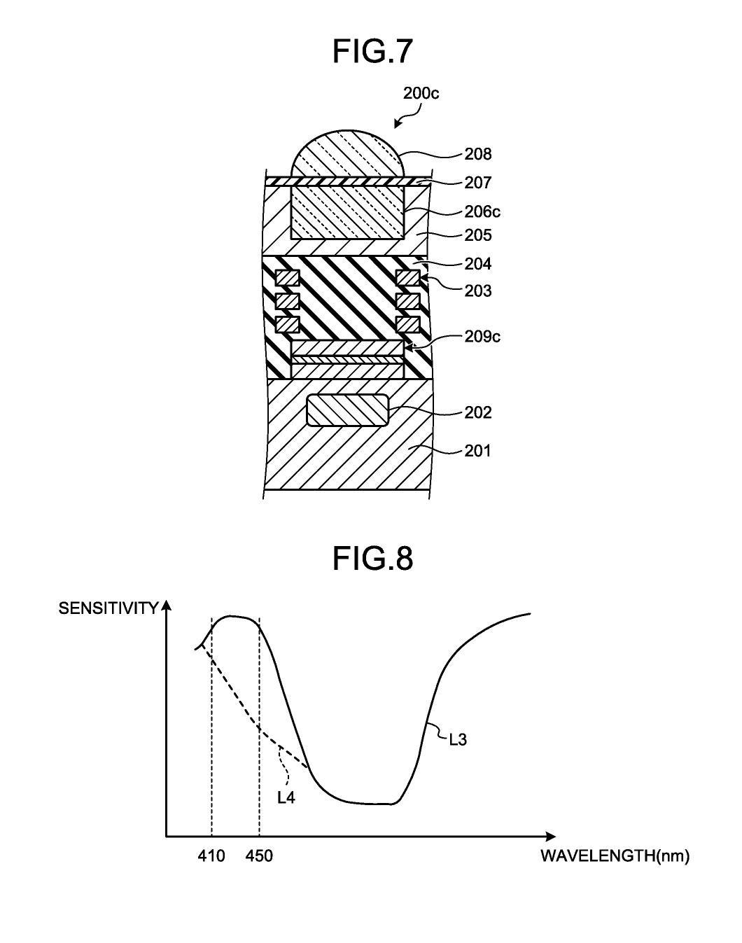

Then, an Mg pixel will be described. FIG. 7 is a sectional view of an Mg pixel. As illustrated in FIG. 7, an Mg pixel 200c includes an Si substrate 201, a photodiode 202 that is formed on the Si substrate 201, a wiring layer 203 that electrically connects pixels, an insulator layer 204 that electrically insulates each wiring layer 203, a buffer layer 205 to planarize a surface, an Mg color filter 206c that is arranged so as to cover the photodiode 202, a protective layer 207 that protects a surface, a microlens 208 formed on an outermost surface, and an Mg multi-layer film 209c as a second multi-layer film disposed on the Si substrate 201.

The Mg color filter 206c is a color filter for passing both of light in a wavelength band of red in the vicinity of 610 nm and light in a wavelength band of blue-violet.

The Mg multi-layer film 209c is a multi-layer film with a refractive index and a layer thickness of each layer being adjusted in such a manner that a peak of reflectivity is between 450 nm and 500 nm.

FIG. 8 is a schematic view illustrating sensitivity of an element including the Mg color filter. A line L3 in FIG. 8 indicates sensitivity of a conventional Mg pixel that includes an Mg color filter 206c and that does not include the Mg multi-layer film 209c. Then, a line L4 (broken line) in FIG. 8 indicates sensitivity of an Mg pixel 200c that includes an Mg color filter 206c and a Mg multi-layer film 209c. That is, under a white light source, light in a wavelength band of red is detected and sensitivity for light in a wavelength band of blue is weakened in the Mg pixel 200c. On the other hand, the Mg pixel 200c detects light in a wavelength band of blue-violet under an NBI illumination light source.

Here, as described with reference to FIG. 3, a G pixel in the Bayer array is replaced with the Cy pixel 200b and an R pixel therein is replaced with the Mg pixel 200c in this endoscope system 1. With this configuration, in the endoscope system 1, all pixels have sensitivity for light in a wavelength band of blue-violet and resolution is improved under an NBI illumination light source. Moreover, in the endoscope system 1, sensitivity for light in a wavelength of each of RGB is included, sensitivity of the Cy pixel 200b and the Mg pixel 200c for light in a wavelength band of blue is weakened, and deterioration in color reproducibility is reduced under the white light source.

It is preferable that light entering the photodiode 202 on which the Cy color filter 206b is disposed and entering the photodiode 202 on which the Mg color filter 206c is disposed has higher intensity in a wavelength band of blue-violet than intensity of light in a wavelength band of blue, by the color filters and multi-layer films. Under this condition, sensitivity for light in the wavelength band of blue-violet under the NBI illumination light is higher than sensitivity for blue light under the white light, which notably reduces deterioration in color reproducibility in normal light imaging while improving sensitivity in NBI.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image sensor comprising:
a plurality of light receiving units disposed two- dimensionally on a substrate and each configured to generate a charge in accordance with an amount of received light;
color filters disposed on the plurality of light receiving units and comprising at least one of:
a blue color filter for passing both of light in a wavelength band of blue and light in a wavelength band of blue-violet;
a cyan color filter for passing both of light in a wavelength band of green and light in the wavelength band of blue-violet; and
a magenta color filter for passing both of light in a wavelength band of red and light in the wavelength band of blue-violet;
a first film arranged on a light receiving unit on which the cyan color filter is disposed, among the plurality of light receiving units, the first film having a peak of reflectivity near 450 nm; and
a second film arranged on a light receiving unit on which the magenta color filter is disposed, among the plurality of light receiving units, the second film having a peak of reflectivity between 450 nm and 500 nm.

2. The image sensor according to claim 1, wherein
the substrate is an Si substrate.

3. The image sensor according to claim 1, wherein
light entering the light receiving unit on which the cyan color filter is disposed and entering the light receiving unit on which the magenta color filter is disposed has intensity in the wavelength band of blue- violet higher than intensity in the wavelength band of blue.

4. The image sensor according to claim 1, wherein
in the color filters,
the cyan color filter and the blue color filter are alternately arranged in an even number line of horizontal lines of the plurality of light receiving units, and
the magenta color filter and the cyan color filter are alternately arranged in an odd number line of the horizontal lines of the plurality of light receiving units.

5. The image sensor according to claim 1, wherein
each of the first film and the second film is a multi- layer film.

6. An imaging device comprising the image sensor according to claim 1.
